(19) United States
(12) Patent Application Publication
(10) Pub. No.: US 2024/0183848 A1
(43) Pub. Date: Jun. 6, 2024

(54) FIRST SUBSTRATE, MICROFLUIDIC CHIP AND METHOD FOR PROCESSING SAMPLE

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Haonan LIU, Beijing (CN); Ding DING, Beijing (CN)

(21) Appl. No.: 17/778,177

(22) PCT Filed: Jun. 8, 2021
(86) PCT No.: PCT/CN2021/098865

§ 371 Date: Dec. 15, 2022

(51) Int. Cl. G01N 33/543; B01L 3/00

(52) U.S. Cl. CPC G01N 33/54386; B01L 3/502761; B01L 2200/16; B01L 2300/0816; B01L 2300/0867; B01L 2300/0883; G01N 2470/04

(57) ABSTRACT

The present application discloses a first substrate for a microfluidic chip, the microfluidic chip, and a method for processing a sample. The first substrate includes a first injection port, a first reacting region, a first upstream end of which is communicated with the first injection port, a second injection port, a second reacting region, an upstream end of which is communicated with the second injection port, and a downstream end of which is communicated with a second upstream end of the first reacting region, a fluid backflow prevention region between the second reacting region and the first reacting region, an upstream end of which is communicated with the downstream end of the second reacting region, and a downstream end of which is communicated with the second upstream end of the first reacting region, and an exit port communicated with a downstream end of the first reacting region.

### FIELD

The present disclosure relates to the field of biological detection, and more particularly to a first substrate for a microfluidic chip, the microfluidic chip, and a method for processing a sample.

### BACKGROUND

Microfluidic chip, also known as Lab-on-a-chip, refers to the integration of basic operating units such as sample preparation, reaction, separation, and detection involved in the fields of biology, chemistry, and medicine into a chip having micron-scale microchannels. It automates the entire process of reaction and analysis. An analytical and detection device based on microfluidic chip can have the advantages of small sample amount, rapid analysis, and very suitable for real time analysis and site analysis. Moreover, the microfluidic chip can be designed as a disposable product, which can eliminate the need for complex fluid channel systems for operations such as cleaning and waste liquid disposal.

### SUMMARY

According to an aspect of this application, there is provided a first substrate for a microfluidic chip. The first substrate comprises a first injection port configured to receive a first fluid; a first reacting region, wherein a first upstream end of the first reacting region is communicated with the first injection port through a flow channel; a second injection port configured to receive a second fluid; a second reacting region, wherein an upstream end of the second reacting region is communicated with the second injection port through a flow channel, and a downstream end of the second reacting region is communicated with a second upstream end of the first reacting region through a flow channel; a fluid backflow prevention region between the second reacting region and the first reacting region, wherein an upstream end of the fluid backflow prevention region is communicated with the downstream end of the second reacting region through a flow channel, and a downstream end of the fluid backflow prevention region is communicated with the second upstream end of the first reacting region through a flow channel; and an exit port communicated with a downstream end of the first reacting region through a flow channel.

In some embodiments, the fluid backflow prevention region comprises: a switching valve on the flow channel between the downstream end of the second reacting region and the second upstream end of the first reacting region.

In some embodiments, the fluid backflow prevention region comprises: a first flow channel extending in a serpentine shape, wherein the first flow channel comprises a plurality of first flow channel subsegments parallel to each other in a plane defined by the first substrate, wherein the plurality of first flow channel subsegments are communicated end to end in sequence through at least one first connection part.

In some embodiments, the first substrate further comprises a first mixing region between the first injection port and the first reacting region, wherein an upstream end of the first mixing region is communicated with the first injection port through a flow channel, and a downstream end of the first mixing region is communicated with the first upstream end of the first reacting region through a flow channel.

In some embodiments, the first mixing region comprises: a second flow channel extending in a serpentine shape, wherein the second flow channel comprises a plurality of second flow channel subsegments parallel to each other in the plane defined by the first substrate, wherein the plurality of second flow channel subsegments are communicated end to end in sequence through at least one second connection part.

In some embodiments, the first substrate further comprises: a second mixing region between the second injection port and the second reacting region, wherein an upstream end of the second mixing region is communicated with the second injection port through a flow channel, and a downstream end of the second mixing region is communicated with the upstream end of the second reacting region through a flow channel.

In some embodiments, the second mixing region comprises: a third flow channel extending in a serpentine shape, wherein the third flow channel comprises a plurality of third flow channel subsegments parallel to each other in the plane defined by the first substrate, wherein the plurality of third flow channel subsegments are communicated end to end in sequence through at least one third connection part.

In some embodiments, the first reacting region comprises a first groove, wherein the first groove comprises a first stage, wherein the first stage is at the first upstream end or the second upstream end of the first reacting region, wherein the first stage and a surface of a non-functional region of the first substrate are separated by a first distance, a remaining part of the first groove except the first stage and the surface of the non-functional region of the first substrate are separated by a second distance, wherein the first distance is less than the second distance, wherein the first groove comprises a first wall, wherein the first wall comprises a first wall segment at the first upstream end or the second upstream end of the first reacting region, the first stage extends a third distance in a direction away from the first wall segment, wherein the first upstream end or the second upstream end of the first reacting region and the downstream end of the first reacting region are separated by a first straight-line distance, and the third distance is between 1/100 of the first straight-line distance and ⅕ of the first straight-line distance.

In some embodiments, the second reacting region comprises a second groove, wherein the second groove comprises a second stage, wherein the second stage is at the upstream end of the second reacting region, wherein the second stage and the surface of the non-functional region of the first substrate are separated by a fourth distance, a remaining part of the second groove except the second stage and the surface of the non-functional region of the first substrate are separated by a fifth distance, wherein the fourth distance is less than the fifth distance, wherein the second groove comprises a second wall, wherein the second wall comprises a second wall segment at the upstream end of the second reacting region, the second stage extends a sixth distance in a direction away from the second wall segment, wherein the upstream end of the second reacting region and the downstream end of the second reacting region are separated by a second straight-line distance, and the sixth distance is between 1/100 of the second straight-line distance and ⅕ of the second straight-line distance.

In some embodiments, a first axis is a straight-line passing through the downstream end of the first reacting region and a midpoint of the first upstream end and the second upstream end of the first reacting region, wherein the first reacting region comprises a first section closer to the first upstream end or the second upstream end of the first reacting region and a second section closer to the downstream end of the first reacting region, wherein an orthographic projection of the first section on a surface of the first substrate is an arch; and wherein the distance between at least one edge of an orthographic projection of the second section on the surface of the first substrate and the first axis gradually decreases along a fluid flow direction.

In some embodiments, a second axis is a straight-line passing through the upstream end of the second reacting region and the downstream end of the second reacting region, wherein the second reacting region comprises a third section closer to the upstream end of the second reacting region and a fourth section closer to the downstream end of the second reacting region, wherein an orthographic projection of the third section on a surface of the first substrate is an arch; and wherein the distance between at least one edge of an orthographic projection of the fourth section on the surface of the first substrate and the second axis gradually decreases along a fluid flow direction.

In some embodiments, the first substrate comprises: a first branch comprising the first injection port, the first mixing region, and a flow channel between the first injection port, the first mixing region and the first upstream end of the first reacting region, and a second branch comprising the second injection port, the second mixing region, the fluid backflow prevention region, the second reacting region, and a flow channel between the second injection port, the second mixing region, the second reacting region, the fluid backflow prevention region and the second upstream end of the first reacting region, wherein a first axis is a straight-line passing through the downstream end of the first reacting region and a midpoint of the first upstream end and the second upstream end of the first reacting region, the first injection port and the second injection port are symmetrically distributed with respect to the first axis, and the first branch and the second branch are respectively located on two sides of the first axis.

In some embodiments, a distance between the first flow channel extending in the serpentine shape and the surface of the non-functional region of the first substrate, a distance between the second flow channel extending in the serpentine shape and the surface of the non-functional region of the first substrate, a distance between the third flow channel extending in the serpentine shape and the surface of the non-functional region of the first substrate, the second distance, and the fifth distance are substantially equal.

In some embodiments, the first flow channel extending in the serpentine shape comprises four first flow channel subsegments parallel to each other in the plane defined by the first substrate, the second flow channel extending in the serpentine shape comprises six second flow channel subsegments parallel to each other in the plane defined by the first substrate, and the third flow channel extending in the serpentine shape comprises five third flow channel subsegments parallel to each other in the plane defined by the first substrate

In some embodiments, a length of the flow channel between the first injection port and the upstream end of the first mixing region is substantially equal to a length of the flow channel between the downstream end of the first mixing region and the first upstream end of the first reacting region.

In some embodiments, the first substrate further comprises a waste liquid region between the first reacting region and the exit port, wherein an upstream end of the waste liquid region is communicated with the downstream end of the first reacting region through a flow channel, and the exit port is at a downstream end of the waste liquid region.

According to another aspect of this application, there is provided a microfluidic chip, comprising: the first substrate according to any one of the embodiments of this application, and a second substrate assembled with the first substrate, wherein the second substrate comprises: a first sample region pre-stored with a capture antibody, and a second sample region pre-stored with a fluorescent antibody, wherein orthographic projections of the first sample region and the second sample region on the first substrate are at least partially overlap with orthographic projections of the first reacting region and the second reacting region on the first substrate, respectively.

In some embodiments, the first substrate comprises a plastic-based material and the second substrate comprises a glass-based material.

According to another aspect of this application, there is provided a method for processing a sample by using the microfluidic chip according to the embodiments of the present application. The method comprises: adding the first fluid through the first injection port, such that the first fluid reacts with the capture antibody in the first reacting region to generate a first product; adding a cleaning liquid through the first injection port, and adjusting a pressure in the flow channel or letting a waste liquid to flow out by the exit port, such that excess impurity in the first substrate is washed away; and adding the second fluid through the second injection port, such that the second fluid reacts in the second reacting region and provides the fluorescent antibody to the first reacting region, and the fluorescent antibody reacts with the first product in the first reacting region to generate a double antibody sandwich compound.

In some embodiments, after the fluorescent antibody reacts with the first product in the first reacting region to generate the double antibody sandwich compound, the method further comprises: adding a buffer liquid through the first injection port, and adjusting the pressure in the flow channel or letting the waste liquid to flow out by the exit port, such that an unreacted fluorescent antibody is washed away.

In some embodiments, after the unreacted fluorescent antibody is washed away, the method further comprises: performing an optical signal detection on the double antibody sandwich compound to determine an antigen content in the sample.

### BRIEF DESCRIPTION OF THE DRAWINGS

In order to illustrate the technical solutions in the embodiments of the present disclosure more clearly, the drawings needed for the description of the embodiments will be briefly introduced below. Apparently, the drawings described below are only some of the embodiments of the present disclosure.

FIG. 1a schematically illustrates a top view of the first substrate according to some embodiments of the present disclosure;

FIGS. 1b-1d schematically illustrate enlarged views of a part of structures of FIG. 1a according to some embodiments of the present disclosure;

FIG. 2 schematically illustrates a cross-sectional view of the first substrate of FIG. 1a along line A-B according to some embodiments of the present disclosure;

FIG. 3 schematically illustrates a cross-sectional view of the first substrate of FIG. 1a along line C-D according to some embodiments of the present disclosure;

FIG. 4 schematically illustrates some exemplary dimensions in the top view of the first substrate of FIG. 1a according to some embodiments of the present disclosure;

FIG. 5 schematically illustrates a simulation diagram of the fluid flow of the first substrate according to some embodiments of the present disclosure;

FIG. 6 schematically illustrates a microfluidic chip according to some embodiments of the present disclosure;

FIGS. 7a-7b schematically illustrate schematic views of the side of a second substrate facing the first substrate according to different embodiments of the present disclosure; and

FIG. 8 schematically illustrates a flow chart of a method for processing a sample according to some embodiments of the present disclosure.

### DETAILED DESCRIPTION OF THE EMBODIMENTS

In order to make the objectives, technical solutions and advantages of the embodiments of the present disclosure clearer, the technical solutions of the embodiments of the present disclosure will be further described in detail below with reference to the accompanying drawings.

It should be understood that, although the terms such as first, second, third, etc. may be used herein to describe various elements, components and/or parts, these elements, components and/or parts should not be limited by these terms. These terms are only used to distinguish one element, component or part from another element, component or part. Thus, a first element, component or part discussed below could also be named as a second element, component or part without departing from the teachings of the present disclosure.

The terminology used herein is only for the purpose of describing particular embodiments, and is not intended to limit the present disclosure. As used herein, the singular articles such as “a”, “an”, and “the” do not intend to exclude the plural forms, unless the context clearly dictates otherwise. It will be further understood that the terms “comprising” and/or “including” when used in this specification indicate the presence of features, entireties, steps, operations, elements and/or components, but do not exclude the presence of one or more other features, entireties, steps, operations, elements, components and/or the groups thereof, or one or more other features, entireties, steps, operations, elements, components and/or the groups thereof can be added. As used herein, the term “and/or” includes any and all combinations of one or more of the associated listed items. As used herein, the term “A or B” refers to at least one of item A and item B, if not contradictory.

In vitro diagnosis industry is mainly divided into biochemical diagnosis, molecular diagnosis and immunodiagnosis according to the principle. In recent years, immunodiagnosis has become the largest subfield of the in vitro diagnosis. Microplates are the main consumables in immunodiagnosis at present. The inventor of the present application found that compared with microplates, microfluidic chips have the advantages of shortened reaction time, reduced reagent consumption, simple operation, integration, automation, portability, etc. It is a new type of immunodiagnosis consumables. The immune microfluidic chip is a biological detection chip. Its principle is to use antigen-antibody specific combination to complete the reaction and show the detection results on the microfluidic chip. Its advantages include that only a small amount of patient specimens or biological samples are needed, and a variety of relevant biological information or disease detection results can be obtained through one detection. Compared with conventional detection methods, it has the advantages of high flux, rapidity, simple operation, and high degree of automation.

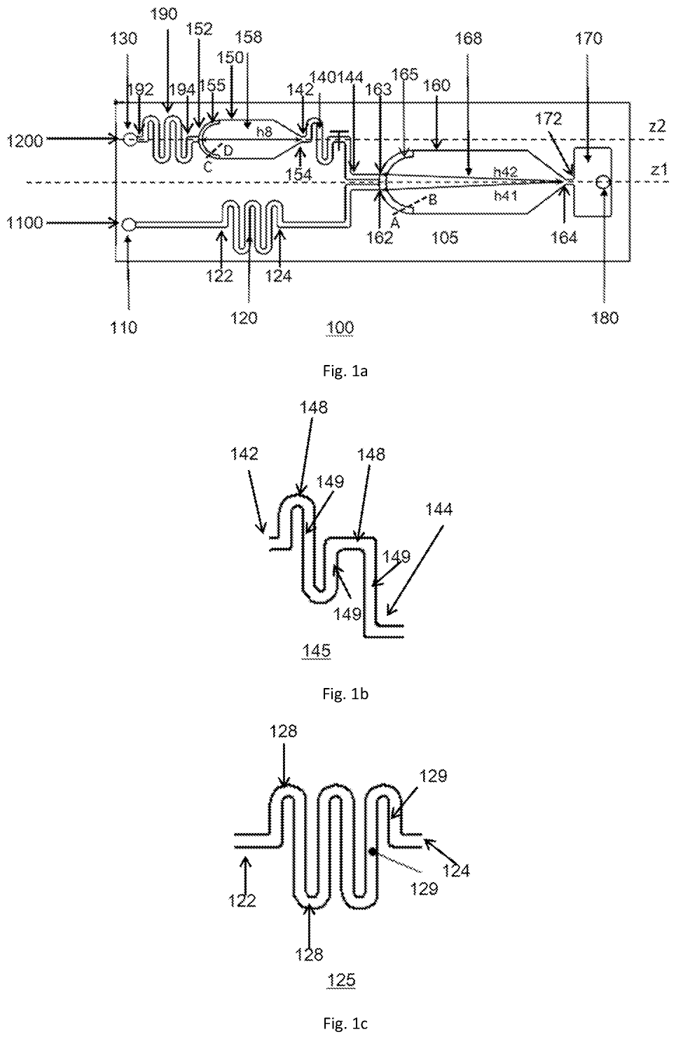

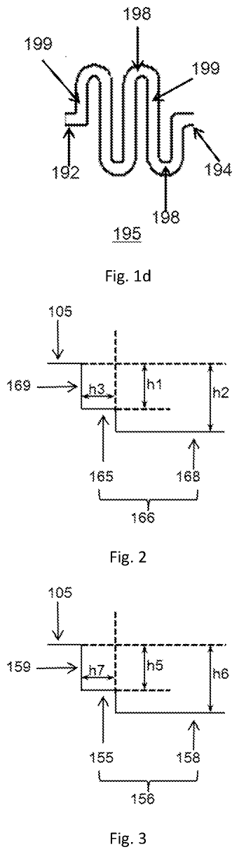

The embodiments of the present application provide a first substrate for a microfluidic chip. FIG. 1a schematically illustrates a top view of a first substrate according to some embodiments of the present disclosure. FIGS. 1b-1d illustrate enlarged views of a part of the structures of FIG. 1a according to some embodiments of the present disclosure. As shown in FIGS. 1a-1d, the first substrate 100 comprises a first injection port 110 configured to receive a first fluid; a first reacting region 160, wherein a first upstream end 162 of the first reacting region 160 is communicated with the first injection port 110 through a flow channel; a second injection port 130 configured to receive a second fluid; a second reacting region 150, wherein an upstream end 152 of the second reacting region 150 is communicated with the second injection port 130 through a flow channel, and a downstream end 154 of the second reacting region 150 is communicated with a second upstream end 163 of the first reacting region 160 through a flow channel; a fluid backflow prevention region 140 between the second reacting region 150 and the first reacting region 160, wherein an upstream end 142 of the fluid backflow prevention region 140 is communicated with the downstream end 154 of the second reacting region 150 through a flow channel and a downstream end 144 of the fluid backflow prevention region 140 is communicated with the second upstream end 163 of the first reacting region 160 through a flow channel; and an exit port 180, communicated with the downstream end 164 of the first reacting region 160 through a flow channel.

It should be understood that the term “upstream end” in this application refers to a port or a terminal of the upstream of the fluid flow direction when the first substrate is operating. For example, the first upstream end of the first reacting region refers to a port of the first reacting region, through which the fluid flows into the first reacting region when the first substrate is operating. It should be understood that the term “downstream end” in this application refers to a port or a terminal of the downstream of the fluid flow direction when the first substrate is operating. For example, the downstream end of the first reacting region refers to a port of the first reacting region, through which the fluid flows out of the first reacting region when the substrate is operating.

It should be understood that the term “flow channel” in this application refers to a space in the first substrate or in the microfluidic chip in which the fluid can flow. For example, the flow channel can be a tubular channel in the microfluidic chip or a grooved channel in the first substrate.

It should be understood that, in some embodiments, the first upstream end 162 of the first reacting region 160 and the second upstream end 163 of the first reacting region 160 are two separate ports. In some embodiments, the first upstream end 162 of the first reacting region 160 and the second upstream end 163 of the first reacting region 160 may overlap to become one port.

The first substrate according to the embodiments of the present disclosure comprises the first reacting region and the second reacting region. The first fluid reacts with the capture antibody in the first reacting region 160 to generate a first product, and the second fluid reacts in the second reacting region 150 and provides the fluorescent antibody to the first reacting region 160, and the fluorescent antibody reacts with the first product in the first reacting region 160 to generate a double antibody sandwich compound. The inventor of the present application found that, in the related art, for the immunoassay microfluidic chip using the principle of immunochromatography (for example, the double antibody sandwich method), the fluorescent antibody is first combined with the antigen, and then the complex of the two is combined with the capture antibody. This combination method is less accurate because the fluorescent antibody combines to the antigen first. In the embodiments of this application, the antigen is first captured by the capture antibody and then combined with the fluorescent antibody. In this way, by using the above-mentioned first substrate, the capture sequence of the antigen and the antibody can be changed. Due to the combination efficiency, such a capture method can more accurately reflect the initial amount of antigen, thereby improving the detection accuracy. The first substrate for the microfluidic chip provided by the embodiments of this application has the advantages of high flux, rapidity, simple operation, and high degree of automation, and the reaction process is optimized in terms of structural design, so that the detection and reaction of the antigen content in the sample is more precise. By providing a fluid backflow prevention region, when the first fluid is received through the first injection port 110 and pumped into the first reacting region 160, the first fluid can be prevented from backflowing into the second reacting region 150. In this way, the reliability and operability of the system are improved, and the cross-contamination between different fluids is prevented, and the accuracy of reaction and detection is improved.

In some embodiments, the fluid backflow prevention region 140 comprises a switching valve. The switching valve is located on a flow channel between the downstream end 154 of the second reacting region 150 and the second upstream end 163 of the first reacting region 160. By providing the switching valve, the conduction and blocking of the flow channel can be controlled to achieve the effect of preventing the fluid backflow.

In some embodiments, the fluid backflow prevention region 140 comprises a first flow channel 145 extending in a serpentine shape. The first flow channel 145 comprises a plurality of first flow channel subsegments 149 parallel to each other in a plane defined by the first substrate 100. The plurality of first flow channel subsegments 149 are communicated end to end in sequence through at least one first connection part 148. For example, the shape of the first connection part 148 may be a semicircular arc or a straight-line, etc. By forming the first flow channel 145 extending in the serpentine shape, the design of the flow channel can be more compact, such that a longer flow channel can be provided on the limited area of the substrate to realize the function of the prevention of fluid backflow of the fluid backflow prevention region 140. It should be understood that the first substrate 100 is a structure whose sizes in two of the three mutually perpendicular dimensions are significantly larger than the size in the rest one dimension. Therefore, it can be understood that the two larger dimensions of the first substrate define a plane. The term “serpentine shape” could be understood as a snakelike shape or a letter S shape, which zigzags like a snake.

In some embodiments, the first substrate 100 further comprises a first mixing region 120 between the first injection port 110 and the first reacting region 160. The upstream end 122 of the first mixing region 120 is communicated with the first injection port 110 through a flow channel, and the downstream end 124 of the first mixing region 120 is communicated with the first upstream end 162 of the first reacting region 160 through a flow channel. By providing the first mixing region, the first fluid can be thoroughly and evenly mixed after entering through the first injection port and before entering into the first reacting region 160. In some embodiments, the first mixing region 120 comprises a second flow channel 125 extending in a serpentine shape. The second flow channel 125 comprises a plurality of the second flow channel subsegments 129 parallel to each other in the plane defined by the first substrate 100. The plurality of the second flow channel subsegments 129 are connected end to end in sequence through at least one second connection part 128. For example, the shape of the second connection part 128 may be a semicircular arc or a straight-line, etc. By providing the second flow channel 125 extending in the serpentine shape, the design of the flow channel can be more compact, such that a longer flow channel can be provided on the limited area of the substrate, so that the first fluid can be mixed more thoroughly and evenly.

In some embodiments, the first substrate 100 further comprises a second mixing region 190 between the second injection port 130 and the second reacting region 150. The upstream end 192 of the second mixing region 190 is communicated with the second injection port 130 through a flow channel, and the downstream end 194 of the second mixing region 190 is communicated with the upstream end 152 of the second reacting region 150 through a flow channel. By providing the second mixing region, the second fluid can be thoroughly and evenly mixed after entering through the second injection port and before entering into the second reacting region 150. In some embodiments, the second mixing region 190 comprises a third flow channel 195 extending in a serpentine shape. The third flow channel 195 comprises a plurality of third flow channel subsegments 199 parallel to each other in the plane defined by the first substrate 100. The plurality of third flow channel subsegments 199 are communicated end to end in sequence through at least one third connection part 198. For example, the shape of the third connection part 198 may be a semicircular arc or a straight-line, etc. By providing the third flow channel 195 extending in the serpentine shape, the design of the flow channel can be more compact, such that a longer flow channel can be provided on a limited area of the substrate, and the first fluid can be mixed more thoroughly and evenly. For example, as shown in FIGS. 1a-1d, the number of the second flow channel subsegments 129 of the first mixing region 120 is greater than the number of the third flow channel subsegments 199 of the second mixing region 190, and the number of the third flow channel subsegments 199 of the second mixing region 190 is greater than the number of the first flow channel subsegments 149 of the fluid backflow prevention region 140. For example, the first flow channel 145 extending in the serpentine shape comprises four first flow channel subsegments 149 parallel to each other in the plane defined by the first substrate 100. The second flow channel 125 extending in the serpentine shape comprises six second flow channel subsegments 129 parallel to each other in the plane defined by the first substrate 100. The third flow channel 195 extending in the serpentine shape comprises five third flow channel subsegments 199 parallel to each other in the plane defined by the first substrate 100. In this way, the length of the flow channel in the first mixing region 120 is greater than the length of the flow channel in the second mixing region 190, and the length of the flow channel in the second mixing region 190 is greater than the length of the flow channel in the fluid backflow prevention region 140. This is set for different fluid mixing and backflow prevention requirements.

FIG. 2 schematically illustrates a cross-sectional view of the first substrate of FIG. 1a along line A-B, according to some embodiments of this application. In some embodiments, as shown in FIG. 1a and FIG. 2, the first reacting region 160 comprises a first groove 166. The first groove 166 comprises a first stage 165. The first stage 165 is provided at the first upstream end 162 or the second upstream end 163 of the first reacting region 160. The first stage 165 and a surface 105 of a non-functional region of the first substrate 100 are separated by a first distance h1. The remaining part 168 of the first groove 166 except the first stage 165 and the surface 105 of the non-functional region of the first substrate 100 are separated by a second distance h2. The first distance h1 is less than the second distance h2. The first groove comprises a first wall 169. The first wall 169 comprises a first wall segment at the first upstream end 162 or the second upstream end 163 of the first reacting region 160. The first stage 165 extends a third distance h3 in a direction away from the first wall segment. In other words, the first stage 165 extends a third distance h3 from the first wall 169 of the first groove 166 close to the first upstream end 162 or the second upstream end 163 of the first reacting region 160. The first upstream end 162 or the second upstream end 163 of the first reacting region 160 and the downstream end 164 of the first reacting region 160 are separated by a first straight-line distance, and the third distance is between 1/100 of the first straight-line distance and ⅕ of the first straight-line distance. In other words, the first upstream end 162 of the first reacting region 160 and the downstream end 164 of the first reacting region 160 are separated by a straight-line distance h41. The second upstream end 163 of the first reacting region 160 and the downstream end 164 of the first reacting region 160 are separated by a straight-line distance h42. h3 is in the range from 1/100 of h41 to ⅕ of h41 or in the range from 1/100 of h42 to ⅕ of h42. By providing the first groove 166 in the first reacting region 160 and providing the first stage 165 in the first groove 166, when a fluid enters the first reacting region 160 through the first upstream end 162 or the second upstream end 163, the fluid will first flow to and spread over the first stage 165, and then flow from the first stage 165 to the remaining part 168 of the first groove 166, making the fluid flow more even and reducing the generation of air bubbles, such that the reaction and the detections are more accurate.

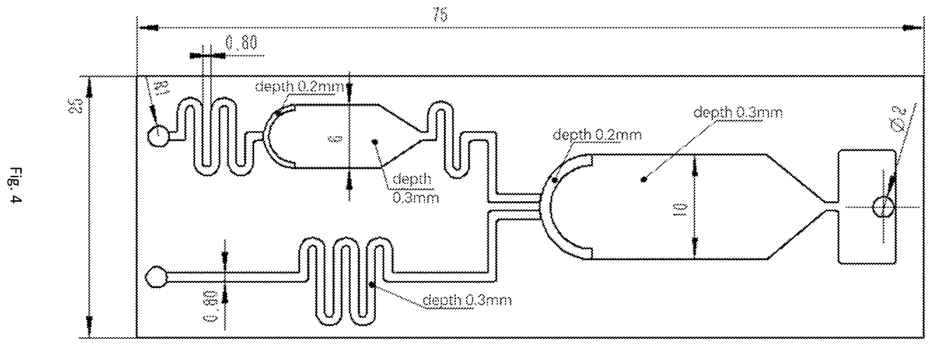

FIG. 3 schematically illustrates a cross-sectional view of the first substrate of FIG. 1 along line C-D, according to some embodiments of this application. In some embodiments, as shown in FIGS. 1a and 3, the second reacting region 150 comprises a second groove 156. The second groove 156 comprises a second stage 155. The second stage 155 is at the upstream end 152 of the second reacting region 150. The second stage 155 and the surface 105 of the non-functional region of the first substrate 100 are separated by a fourth distance h5. A remaining part 158 of the second groove 156 except the second stage 155 and the surface 105 of the non-functional region of the first substrate 100 are separated by a fifth distance h6. The fourth distance h5 is less than the fifth distance h6. The second groove 156 comprises a second wall 159. The second wall 159 comprises a second wall segment at the upstream end 152 of the second reacting region 150. The second stage 155 extends a sixth distance h7 in a direction away from the second wall segment. In other words, the second stage 155 extends a sixth distance h7 from the wall 159 of the second groove 156 close to the upstream end 152 of the second reacting region 150. The upstream end 152 of the second reacting region 150 and the downstream end 154 of the second reacting region 150 are separated by a second straight-line distance h8, and the sixth distance h7 is between 1/100 of the second straight-line distance h8 and ⅕ of the second straight-line distance h8. In other words, h7 is in the range from 1/100 of h8 to ⅕ of h8. By providing the second groove 156 in the second reacting region 150 and providing the second stage 155 in the second groove 156, when a fluid enters the second reacting region 150 through the upstream end 152, the fluid will first flow to and spread over the second stage 155, and then flow from the second stage 155 to the remaining part 158 of the second groove 156, making the fluid flow more even and reducing the generation of air bubbles, such that the reaction and detection are more accurate.

In some embodiments, referring to FIG. 1a, a first axis z1 is a straight-line passing through the downstream end 164 of the first reacting region 160 and the midpoint of the first upstream end 162 and the second upstream end 163 of the first reacting region 160. The first reacting region 160 comprises a first section closer to the first upstream end 162 or the second upstream end 163 of the first reacting region 160. The first reacting region 160 further comprises a second section closer to the downstream end 164 of the first reacting region 160. An orthographic projection of the first section on a surface of the first substrate 100 is an arch. In other words, in a part of the first reacting region 160 closer to the first upstream end 162 or the second upstream end 163, the orthographic projection of the first reacting region 160 on the surface of the first substrate 100 is in an arch shape. The distance between at least one edge of an orthographic projection of the second section on the surface of the first substrate 100 and the first axis z1 gradually decreases along a fluid flow direction. In other words, in a part of the first reacting region 160 closer to the downstream end 164, the orthographic projection of the first reacting region 160 on the surface of the first substrate 100 tapers along the fluid flow direction. By providing such a shape, the fluid flows more evenly and the generation of air bubbles is reduced, such that the reaction and detection are more accurate.

In some embodiments, referring to FIG. 1a, a second axis z2 is a straight-line passing through the upstream end 152 of the second reacting region 150 and the downstream end 154 of the second reacting region 150. The second reacting region 150 comprises a third section closer to the upstream end 152 of the second reacting region 150 and a fourth section closer to the downstream end 154 of the second reacting region 150. An orthographic projection of the third section on a surface of the first substrate 100 is an arch. In other words, in a part of the second reacting region 150 closer to the upstream end 152, the orthographic projection of the second reacting region 150 on the surface of the first substrate 100 is in an arch shape. The distance between at least one edge of an orthographic projection of the fourth section on the surface of the first substrate 100 and the second axis z2 gradually decreases along a fluid flow direction. In other words, in a part of the second reacting region 150 closer to the downstream end 154, the orthographic projection of the second reacting region 150 on the surface of the first substrate 100 tapers along the fluid flow direction. By providing such a shape, the fluid flows more even and the generation of air bubbles is reduced, such that the reaction and detection are more accurate.

In some embodiments, the first substrate 100 comprises a first branch 1100 comprising the first injection port 110, the first mixing region 120, and a flow channel between the first injection port 110, the first mixing region 120 and the first upstream end 162 of the first reacting region. The first substrate 100 further comprises a second branch 1200 comprising the second injection port 130, the second mixing region 190, the fluid backflow prevention region 140, the second reacting region 150, and a flow channel between the second injection port 130, the second mixing region 190, the second reacting region 150, the fluid backflow prevention region 140 and the second upstream end 163 of the first reacting region. The first axis z1 is a straight-line passing through the downstream end 164 of the first reacting region and the midpoint of the first upstream end 162 and the second upstream end 163 of the first reacting region. The first injection port 110 and the second injection port 130 are symmetrically distributed with respect to the first axis z1, and the first branch 1100 and the second branch 1200 are on two sides of the first axis z1, respectively. In this way, the first branch and the second branch can respectively be distributed on one side and the other side of the first substrate, which optimizes the spatial layout of the first substrate, saves the space, and improves the operation effect of different functional regions and flow channels.

In some embodiment, a distance between the first flow channel 145 extending in the serpentine shape and the surface 105 of the non-functional region of the first substrate 100, a distance between the second flow channel 125 extending in the serpentine shape and the surface 105 of the non-functional region of the first substrate 100, a distance between the third flow channel 195 extending in the serpentine shape and the surface 105 of the non-functional region of the first substrate 100, the second distance, and the fifth distance are substantially equal. In this way, the spatial layout of the first substrate is optimized, the processing is facilitated, the space is saved, and the operation effect of different functional regions and flow channels is improved.

In some embodiment, a length of the flow channel between the first injection port 110 and the upstream end 122 of the first mixing region 120 is substantially equal to a length of the flow channel between the downstream end 124 of the first mixing region 120 and the first upstream end 162 of the first reacting region 160. In this way, the spatial layout of the first substrate is optimized, the mixing effect of the first mixing region is improved, the space is saved, and the operation effect of different functional regions and flow channels is optimized.

It should be understood that, in the present disclosure, the expression such as A and B being “substantially equal” indicates that A and B are approximately equal or roughly equal. For example, A is between 80%-120% of B, 90-110% of B, or 95%-105% of B, or B is between 80%-120% of A, between 90-110% of A, or between 95%-105% of A, etc. In some embodiments, referring to FIG. 1a, the first substrate 100 further comprise a waste liquid region 170 between the first reacting region 160 and the exit port 180. An upstream end 172 of the waste liquid region 170 is communicated with the downstream end 164 of the first reacting region 160 through a flow channel, and an exit port 180 is at the downstream end of the waste liquid region 170. The waste liquid region is used to collect and accommodate the waste liquid generated during the operation, so that all waste liquid can be discharged in one time from the exit port 180 after the entire reaction is completed. In some embodiments, the exit port 180 is only used for venting or regulating the pressure, and the waste liquid is collected and accommodated in the waste liquid region 170.

FIG. 4 schematically illustrates some exemplary dimensions in the top view of the first substrate of FIG. 1a according to some embodiments of the present application. Some exemplary dimensions in the top view of the first substrate 100 in some embodiments are described below with reference to FIGS. 1a and 4. For example, the first substrate 100 has a length of 50-100 mm, such as 75 mm, and a width of 20-30 mm, such as 25 mm. For example, the width of the flow channel between the first injection port 110 and the first mixing region 120 is 0.50-1.0 mm, such as 0.80 mm. For example, the distance between the at least one second flow channel subsegments comprised by the first mixing region 120 and the surface 105 of the non-functional region of the first substrate 100 is 0.20-0.40 mm, such as 0.30 mm. For example, the distance between two adjacent third flow channel subsegments comprised by the second mixing region 190 is 0.50-1.0 mm, for example, 0.80 mm. For example, the radius R1 of the second injection port 130 is 0.50-1.5 mm, such as 0.80 mm. For example, the distance between the upper wall and the lower wall of the first groove 166 is 8-15 mm, such as 10 mm. For example, the distance between the upper wall and the lower wall of the second groove 156 is 5-10 mm, such as 6 mm. For example, the first distance h1 between the first stage 165 and the surface 105 of the non-functional region of the first substrate 100 is 0.2 mm, and the second distance h2 between the remaining part 168 of the first groove 166 and the surface 105 of the non-functional region of the first substrate 100 is 0.3 mm. For example, the fourth distance h5 between the second stage 155 and the surface 105 of the non-functional region of the first substrate 100 is 0.2 mm, and the fifth distance h6 between the remaining part 158 of the second groove 150 and the surface 105 of the non-functional region of the first substrate 100 is 0.3 mm. For example, the diameter of the exit port 180 may be 1-3 mm, such as 2 mm.

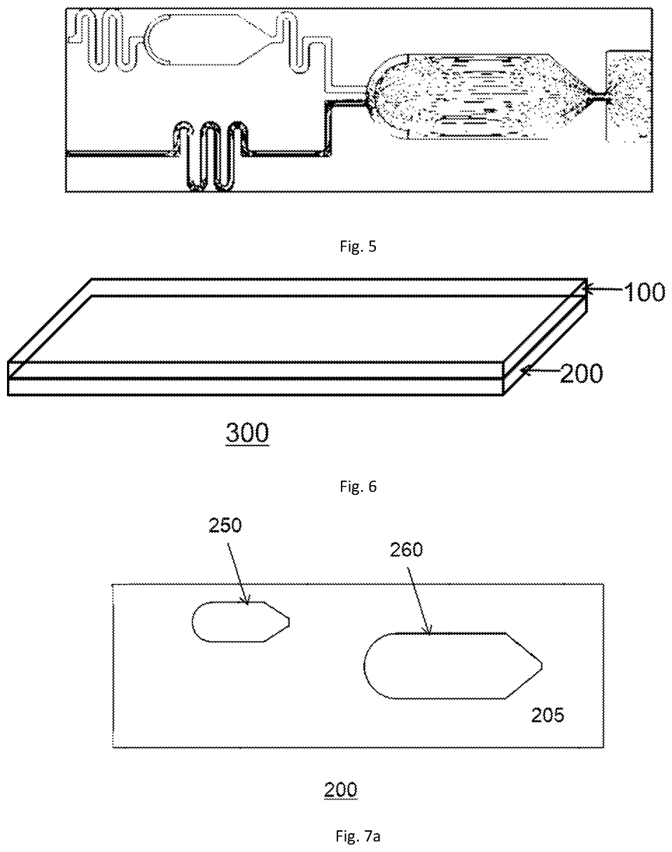

FIG. 5 schematically illustrates a simulation diagram of the fluid flow of the first substrate according to some embodiments of the present disclosure. As shown in FIG. 5, when the first fluid is received through the first injection port 110 and pumped into the first reacting region 160 and the waste liquid region 170, the first fluid hardly flows back into the second reacting region 150. The first substrate has good reliability and operability, and prevents cross-contamination between different fluids, and improves the accuracy of reactions and detection.

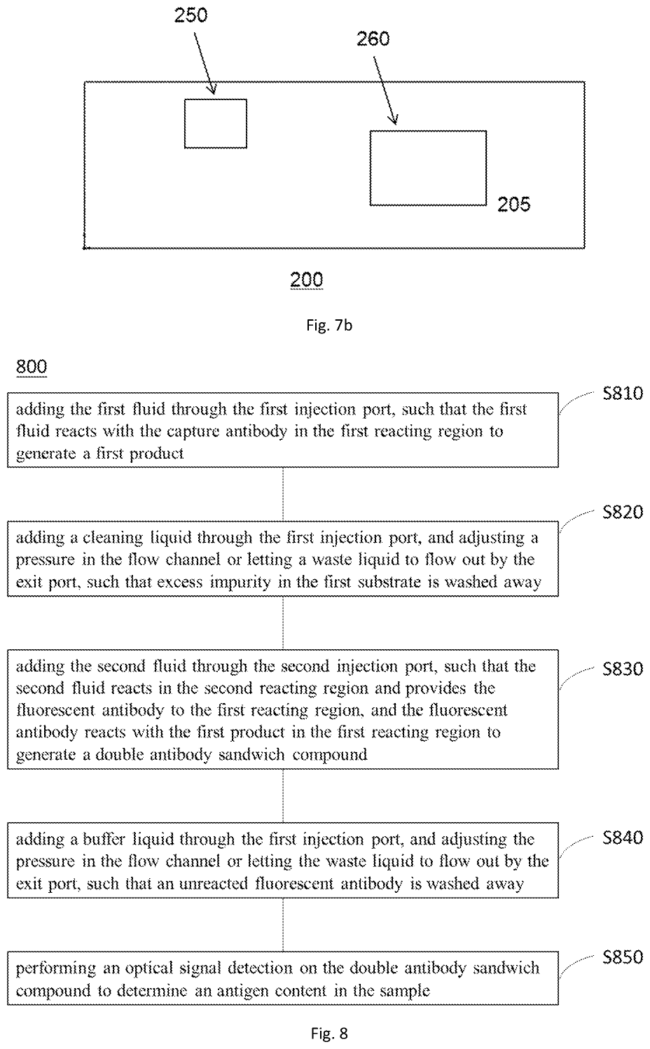

In another aspect of the present application, there is provided a microfluidic chip. FIG. 6 schematically illustrates a microfluidic chip 300 according to some embodiments of the present disclosure. FIGS. 7a-7b schematically illustrate schematic views of the side of the second substrate facing the first substrate according to different embodiments of the present disclosure. The microfluidic chip 300 comprises the first substrate 100 described above, and a second substrate 200 assembled with the first substrate 100. The second substrate 200 comprises a first sample region 260 pre-stored with the capture antibody and a second sample region 250 pre-stored with the fluorescent antibody. FIGS. 7a-7b schematically illustrate different shapes of the first sample region 260 and the second sample region 250 according to various embodiments of the present disclosure. For example, as shown in FIG. 7a, the first sample region 260 and the second sample region 250 may have substantially the same shape as the first reacting region 160 and the second reacting region 150, respectively, and the orthographic projections of the first sample region 260 and the second sample region 250 on the first substrate at least partially overlap with the orthographic projections of the first reacting region 160 and the second reacting region 150 on the first substrate, respectively. For example, as shown in FIG. 7b, the first sample region 260 and the second sample region 250 may have substantially rectangular shapes, respectively, and the orthographic projections of the first sample region 260 and the second sample region 250 on the first substrate at least partially overlap with the orthographic projections of the first reacting region 160 and the second reacting region 150 on the first substrate, respectively.

By providing the first substrate 100 and the second substrate 200 assembled with the first substrate 100, a microfluidic chip 300 is formed. By providing the first sample region 260 and the second sample region 250, the positions of which corresponding to the first reacting region 160 and the second reacting region 150, respectively, the capture antibody and the fluorescent antibody required by the reaction in the microfluidic chip can be pre-stored on the second substrate, so that there is no need to provide antibodies directly on the first substrate, which makes the selection of the material of the first substrate more extensive and easy to process.

In some embodiments, the first substrate comprises a plastic-based material, and the second substrate comprises a glass-based material. For example, the second substrate of the chip is pure glass on which silicon dioxide sample regions (the first sample region 260 and the second sample region 250) are deposited. The first substrate uses the plastic-based material (such as PMMA), such that the flow channels and the reacting regions are easier to be manufactured. The material of the second substrate can be a glass-based material, because the chemical modification of the glass-based material is better than that of the plastic-based material. After the chemical modification of the glass-based material is completed, the antibody can be grafted. In addition, a variety of capture antibodies can also be pre-stored on the first sample region 260 of the glass-based material through a sample applicator, in order to achieve the effect of multi-index joint inspection. Compared with the conventional immunoassay microfluidic chip, the first substrate in this application can be made of a plastic-based material, so that the flow channels and reacting regions required for the immune reaction is easy to produce and the assemble of the substrates is facilitated. The flow channels and reacting regions are more difficult to be prepared on a glass-based material. The second substrate can store and immobilize the antibodies for the reaction. The second substrate can be a glass-based material, in order to use the advantages of chemical modification of the glass-based material.

In some embodiments, each functional region and each flow channel of the first substrate 100 are open spaces that are open to the air. The second substrate 200 is in a regular plate shape (provided with through holes for the first injection port 110, the second injection port 130 and the exit port 180, and the first sample region 260 and the second sample region 250). By the assemble of the first substrate 100 and the second substrate 200, the open spaces of the first substrate 100 are transferred to flow channels for fluid flow. In some embodiments, when the microfluidic chip 300 is operating, as shown in FIG. 6, the second substrate 200 is below the first substrate 100. In some embodiments, the second substrate 200 and the first substrate 100 may be assembled with double-sided tape. For example, after die-cutting (i.e., cutting according to the shape of the flow channels and the functional regions) a double-sided tape with a thickness of several tens of microns, the second substrate 200 and the first substrate 100 are attached and packaged by using the double-sided tape.

The microfluidic chip may have similar features or advantages as the first substrate described above, which will not be repeated here.

In yet another aspect, the embodiments of the present application further provide a method for processing a sample using the above-mentioned microfluidic chip. FIG. 8 schematically illustrates a flow chart of the method 800 for processing a sample according to some embodiments of the present disclosure. The method 800 for processing a sample comprises the following steps S810-S830.

S810: adding the first fluid through the first injection port, such that the first fluid reacts with the capture antibody in the first reacting region to generate a first product.

For example, the first fluid (the sample to be tested) enters through the first injection port 110, and after passing through the first mixing region 120 for mixing, the first fluid enters into the first reacting region 160. A capture antibody is pre-stored at a position (the first sample region) of the second substrate corresponding to the first reacting region 160. The antigen to be tested in the sample reacts with the capture antibody pre-stored in the corresponding glass substrate to generate the first product (the antigen-antibody complex). Due to the design of the fluid backflow prevention region 140, only a very small amount of the fluid pumped from the first injection port 110 may enter the second reacting region 150.

S820: adding a cleaning liquid through the first injection port, and adjusting a pressure in the flow channel or letting the waste liquid to flow out by the exit port, such that excess impurity in the first substrate is washed away.

S830: adding a second fluid through the second injection port, such that the second fluid reacts in the second reacting region and provides the fluorescent antibody to the first reacting region, and the fluorescent antibody reacts with the first product in the first reacting region to generate a double antibody sandwich compound.

For example, the second fluid (the redissolve liquid) is pumped in through the second injection port 130. A lyophilized fluorescent antibody is pre-stored in the position of the second substrate (the second sample region) corresponding to the second reacting region 150. The redissolve liquid redissolves the lyophilized fluorescent antibody pre-stored in the second sample region, and causes the fluorescent antibody to enter the first reacting region 160 and reacts with the first product (the antigen-antibody complex) in the first reacting region 160 to form a double antibody sandwich compound.

In some embodiments, after the fluorescent antibody reacts with the first product in the first reacting region to generate the double antibody sandwich compound, the method further comprises S840: adding a buffer liquid through the first injection port, and adjusting the pressure in the flow channel or letting the waste liquid to flow out by the exit port, such that the unreacted fluorescent antibody is washed away.

In some embodiments, after the unreacted fluorescent antibody is washed away, the method 800 further comprises S850: performing an optical signal detection on the double antibody sandwich compound to determine an antigen content in the sample. For example, the microfluidic chip is placed under a fluorescence microscope for the optical signal detection, so as to determine the antigen content in the sample.

The method for processing a sample provided in the embodiments of the present application has the similar features or advantages as the first substrate and the microfluidic chip described above, and will not be repeated here.

As will be apparent to those skilled in the art, many different ways of implementing the method of these disclosed embodiments are possible. For example, the order of the steps may be changed, or some steps may be performed in parallel. Furthermore, other method steps may be inserted between the steps. The inserted steps may represent such as the improvements to the method described herein, or may be unrelated to the method. Also, a given step may not be fully completed before the next step begins. It should be understood that features of different embodiments of this disclosure may be used in combination with each other, provided that they do not contradict each other.

Various modifications and variations can be made to the present disclosure by those skilled in the art without departing from the spirit and scope of the present disclosure. Thus, if these modifications and variations of the present disclosure fall within the scope of the claims of the present disclosure and their equivalents, the present disclosure is also intended to cover such modifications and variations.

1. A first substrate for a microfluidic chip, comprising:
a first injection port configured to receive a first fluid;
a first reacting region, wherein a first upstream end of the first reacting region is communicated with the first injection port through a flow channel;
a second injection port configured to receive a second fluid;
a second reacting region, wherein an upstream end of the second reacting region is communicated with the second injection port through a flow channel, and a downstream end of the second reacting region is communicated with a second upstream end of the first reacting region through a flow channel;
a fluid backflow prevention region between the second reacting region and the first reacting region, wherein an upstream end of the fluid backflow prevention region is communicated with the downstream end of the second reacting region through a flow channel, and a downstream end of the fluid backflow prevention region is communicated with the second upstream end of the first reacting region through a flow channel; and
an exit port communicated with a downstream end of the first reacting region through a flow channel.

2. The first substrate according to claim 1, wherein the fluid backflow prevention region comprises:
a switching valve on the flow channel between the downstream end of the second reacting region and the second upstream end of the first reacting region.

3. The first substrate according to claim 1, wherein the fluid backflow prevention region comprises:
a first flow channel extending in a serpentine shape, wherein the first flow channel comprises a plurality of first flow channel subsegments parallel to each other in a plane defined by the first substrate,
wherein the plurality of first flow channel subsegments are communicated end to end in sequence through at least one first connection part.

4. The first substrate according to claim 3, further comprising:
a first mixing region between the first injection port and the first reacting region, wherein an upstream end of the first mixing region is communicated with the first injection port through a flow channel, and a downstream end of the first mixing region is communicated with the first upstream end of the first reacting region through a flow channel.

5. The first substrate according to claim 4, wherein the first mixing region comprises:
a second flow channel extending in a serpentine shape, wherein the second flow channel comprises a plurality of second flow channel subsegments parallel to each other in the plane defined by the first substrate,
wherein the plurality of second flow channel subsegments are communicated end to end in sequence through at least one second connection part.

6. The first substrate according to claim 5, further comprising:
a second mixing region between the second injection port and the second reacting region, wherein an upstream end of the second mixing region is communicated with the second injection port through a flow channel, and a downstream end of the second mixing region is communicated with the upstream end of the second reacting region through a flow channel.

7. The first substrate according to claim 6, wherein the second mixing region comprises:
a third flow channel extending in a serpentine shape, wherein the third flow channel comprises a plurality of third flow channel subsegments parallel to each other in the plane defined by the first substrate,
wherein the plurality of third flow channel subsegments are communicated end to end in sequence through at least one third connection part.

8. The first substrate according to claim 7, wherein the first reacting region comprises a first groove,
wherein the first groove comprises a first stage, wherein the first stage is at the first upstream end or the second upstream end of the first reacting region,
wherein the first stage and a surface of a non-functional region of the first substrate are separated by a first distance, a remaining part of the first groove except the first stage and the surface of the non-functional region of the first substrate are separated by a second distance,
wherein the first distance is less than the second distance,
wherein the first groove comprises a first wall,
wherein the first wall comprises a first wall segment at the first upstream end or the second upstream end of the first reacting region, the first stage extends a third distance in a direction away from the first wall segment, and
wherein the first upstream end or the second upstream end of the first reacting region and the downstream end of the first reacting region are separated by a first straight-line distance, and the third distance is between 1/100 of the first straight-line distance and ⅕ of the first straight-line distance.

9. The first substrate according to claim 8, wherein the second reacting region comprises a second groove,
wherein the second groove comprises a second stage, wherein the second stage is at the upstream end of the second reacting region,
wherein the second stage and the surface of the non-functional region of the first substrate are separated by a fourth distance, a remaining part of the second groove except the second stage and the surface of the non-functional region of the first substrate are separated by a fifth distance,
wherein the fourth distance is less than the fifth distance,
wherein the second groove comprises a second wall,
wherein the second wall comprises a second wall segment at the upstream end of the second reacting region, the second stage extends a sixth distance in a direction away from the second wall segment, and
wherein the upstream end of the second reacting region and the downstream end of the second reacting region are separated by a second straight-line distance, and the sixth distance is between 1/100 of the second straight-line distance and ⅕ of the second straight-line distance.

10. The first substrate according to claim 1, wherein a first axis is a straight-line passing through the downstream end of the first reacting region and a midpoint of the first upstream end and the second upstream end of the first reacting region,
wherein the first reacting region comprises a first section closer to the first upstream end or the second upstream end of the first reacting region and a second section closer to the downstream end of the first reacting region,
wherein an orthographic projection of the first section on a surface of the first substrate is an arch, and
wherein a distance between at least one edge of an orthographic projection of the second section on the surface of the first substrate and the first axis gradually decreases along a fluid flow direction.

11. The first substrate according to claim 1,
wherein a second axis is a straight-line passing through the upstream end of the second reacting region and the downstream end of the second reacting region,
wherein the second reacting region comprises a third section closer to the upstream end of the second reacting region and a fourth section closer to the downstream end of the second reacting region,
wherein an orthographic projection of the third section on a surface of the first substrate is an arch; and
wherein a distance between at least one edge of an orthographic projection of the fourth section on the surface of the first substrate and the second axis gradually decreases along a fluid flow direction.

12. The first substrate according to claim 6, comprising:
a first branch comprising the first injection port, the first mixing region, and a flow channel between the first injection port, the first mixing region and the first upstream end of the first reacting region, and
a second branch comprising the second injection port, the second mixing region, the fluid backflow prevention region, the second reacting region, and a flow channel between the second injection port, the second mixing region, the second reacting region, the fluid backflow prevention region and the second upstream end of the first reacting region,
wherein a first axis is a straight-line passing through the downstream end of the first reacting region and a midpoint of the first upstream end and the second upstream end of the first reacting region, the first injection port and the second injection port are symmetrically distributed with respect to the first axis, and the first branch and the second branch are respectively located on two sides of the first axis.

13. The first substrate according to claim 9, wherein a distance between the first flow channel extending in the serpentine shape and the surface of the non-functional region of the first substrate, a distance between the second flow channel extending in the serpentine shape and the surface of the non-functional region of the first substrate, a distance between the third flow channel extending in the serpentine shape and the surface of the non-functional region of the first substrate, the second distance, and the fifth distance are substantially equal.

14. (canceled)

15. The first substrate according to claim 4, wherein a length of the flow channel between the first injection port and the upstream end of the first mixing region is substantially equal to a length of the flow channel between the downstream end of the first mixing region and the first upstream end of the first reacting region.

16. The first substrate according to claim 1, further comprising a waste liquid region between the first reacting region and the exit port, wherein an upstream end of the waste liquid region is communicated with the downstream end of the first reacting region through a flow channel, and the exit port is at a downstream end of the waste liquid region.

17. A microfluidic chip, comprising:
the first substrate according to claim 1, and
a second substrate assembled with the first substrate,
wherein the second substrate comprises:
a first sample region pre-stored with a capture antibody, and
a second sample region pre-stored with a fluorescent antibody,
wherein orthographic projections of the first sample region and the second sample region on the first substrate are at least partially overlap with orthographic projections of the first reacting region and the second reacting region on the first substrate, respectively.

18. The microfluidic chip according to claim 17, wherein the first substrate comprises a plastic-based material and the second substrate comprises a glass-based material.

19. A method for processing a sample by using the microfluidic chip according to claim 17, comprising:
adding the first fluid through the first injection port, such that the first fluid reacts with the capture antibody in the first reacting region to generate a first product;
adding a cleaning liquid through the first injection port, and adjusting a pressure in the flow channel or letting a waste liquid to flow out by the exit port, such that excess impurity in the first substrate is washed away; and
adding the second fluid through the second injection port, such that the second fluid reacts in the second reacting region and provides the fluorescent antibody to the first reacting region, and the fluorescent antibody reacts with the first product in the first reacting region to generate a double antibody sandwich compound.

20. The method according to claim 19, wherein after the fluorescent antibody reacts with the first product in the first reacting region to generate the double antibody sandwich compound, the method further comprises:
adding a buffer liquid through the first injection port, and adjusting the pressure in the flow channel or letting the waste liquid to flow out by the exit port, such that an unreacted fluorescent antibody is washed away.

21. The method according to claim 20, wherein after the unreacted fluorescent antibody is washed away, the method further comprises:
performing an optical signal detection on the double antibody sandwich compound to determine an antigen content in the sample.
